(19) United States
(12) Patent Application Publication
(10) Pub. No.: US 2012/0022568 A1
(43) Pub. Date: Jan. 26, 2012

(54) Instrument for Use in Bone and Method of Use

(75) Inventors: Antony Koblish, Malvern, PA (US); Jeffrey G. Marx, Chester Springs, PA (US); Theodore D. Clineff, Phoenixville, PA (US); Stephen G. Gara, Souderton, PA (US); Troy Wilford, Romansville, PA (US); Paul Tashjian, Phoenixville, PA (US); Maarten Persenaire, Phoenixville, PA (US); Dean Entrekin, Red Bank, NJ (US); Erik M. Erbe, Rancho Santa Fe, CA (US)

(73) Assignee: ORTHOVITA, INC.

(21) Appl. No.: 13/035,650

(22) Filed: Feb. 25, 2011

Related U.S. Application Data
(60) Provisional application No. 61/310,039, filed on Mar. 3, 2010.

(51) Int. Cl. A61B 17/34

(52) U.S. Cl. 606/185

(57) ABSTRACT

Instrument for creating voids and channels in bone; and for obtaining samples of bone tissue. The instruments are suitable for reducing fractures in bone and for compacting the bone to create a barrier for leakage upon material injection. The instruments may also be used for bone biopsy and for obtaining samples of bone tissue. Methods for using these instruments are also disclosed.

### TECHNICAL FIELD

The present invention relates generally to instruments for use in bone. In particular, the present invention relates to instruments for creating voids and channels in bone. The present invention also relates to the use of instruments for reducing fractures in bone. The present invention further relates to the use of instruments for bone biopsy and instruments for obtaining samples of bone tissue.)

### BACKGROUND

Various medical procedures require the reduction of a fracture in bone, the creation of a void, channel or cavity in bone and/or the removal of a bone tissue sample. Among these procedures are vertebroplasty, kyphoplasty, sacroplasty, osteoplasty, and bone biopsy. Vertebroplasty is a procedure for the treatment of vertebral compression fractures. In a typical vertebroplasty procedure, the patient is treated with local anesthesia and light sedation, usually in an x-ray suite or operating room on an outpatient basis. A cannula or needle is guided into the fractured vertebra under x-ray guidance through a small puncture in the patient's skin. A tamp, trocar, stylet or micro-reamer may then be used to reduce the fracture and/or create a channel in the bone to make way for bone cement or bone augmentation material. Upon creation of a channel, cement or bone augmentation material is injected into the fractured vertebra via the needle or via the use of a catheter, tubing and/or syringe, to stabilize the vertebral body. Kyphoplasty is an alternate procedure for the treatment of vertebral compression fractures in which one or more inflatable balloon tamps are used to create a large void or cavity in the vertebral body to restore the height and shape of the vertebral body. Cavity creation is followed by the injection of bone cement or bone augmentation material to strengthen the vertebral body. Sacroplasty is a procedure for the treatment of pelvic (sacral) fractures in which the fracture is internally cast with bone cement material. Osteoplasty is a procedure for the surgical treatment or repair of bone.

Surgical instruments for creating channels, voids and cavities and for cutting bone tissue are well known. Instruments that remove this compacted, compressed and/or cut tissue from the body are also well known. For example, instruments such as drills, ronguers, needles, curettes, trocars, reamers and the like are known for their use in creating voids in bone and for removing samples of the bone. See for instance, U.S. Pat. Nos. 6,679,886; 6,726,691; 6,716,216; and U.S. Patent Application Numbers 2007/0282345; 2007/0203500; 2007/0123889; 200710068329; 2006/0241644.

However, many of the instruments of the prior art are bulky and not practical for minimally invasive/percutaneous procedures. Conversely, smaller instruments suitable for minimally invasive procedures are often delicate. Furthermore, the use of these instruments is often secondary to the primary procedure and therefore their use lengthens the surgical procedure time by creating a secondary procedure.

Accordingly, there is a need in the art for durable instruments that can be used in conjunction with other tools and instruments used in primary procedures, including minimally invasive/percutaneous procedures, to streamline the surgical procedure. Furthermore, there is a need in the art for instruments that reduce fractures in bone and produce cavities and voids in bone in a reliable manner. There is also a need for instruments that can create cavities and voids in bone of varying quality including normal cortical and cancellous bone as well as osteoporotic bone.

### SUMMARY

The present invention is directed to instruments for creating voids and channels in bone. The present invention is also directed to instruments for reducing fractures in bone and for compacting the bone to create a barrier for leakage upon material injection. The present invention further relates to the use of instruments for bone biopsy and instruments for obtaining samples of bone tissue. The present invention is also directed to methods for using these instruments.

### BRIEF DESCRIPTION OF SEVERAL VIEWS OF THE DRAWINGS

The invention is best understood from the following detailed description when read in connection with the accompanying figures. It is emphasized that, according to common practice, the various features of the figures are not to scale. On the contrary, the dimensions of the various features are arbitrarily expanded or reduced for clarity. Included are the following figures:

FIG. 1A is a schematic of one embodiment of the present invention showing the distal end of the cannula/needle with opposing slits.

FIG. 1B is a schematic of the embodiment of FIG. 1A showing at least one bump located on the inside surface of the hollow lumen, protruding into the lumen.

FIGS. 1C-1E are schematics of the embodiment of FIG. 1A showing the stylet that cooperates with the embodiment of FIG. 1A/1B. The stylet may have flattened parallel surfaces (FIG. 1D) or alternatively grooves (FIG. 1E) instead of flattened surfaces for mating with the bumps/protrusions of the cannula/needle.

FIG. 1F is a schematic of the embodiment of FIG. 1A showing that actuation (via rotation) of the stylet causes the outer surface of the stylet to engage the bumps/protrusions on the inside of the lumen of the cannula/needle thereby causing the distal end of the cannula/needle to flare.

FIG. 1G is a schematic showing the proximal nested handles of the stylet and cannula/needle of the embodiment of FIG. 1A.

FIG. 2A is an image of another embodiment of the present invention showing a cannula/needle with at least one slot at its distal end.

FIG. 2B is a cross-sectional image showing the modular component within the hollow lumen of the cannula/needle of FIG. 2A. The modular component is in the fixed position with the tubular guide member and the cutting element nested together.

FIG. 2C is a perspective image showing the modular component within the hollow lumen of the cannula/needle of FIG. 2A. The modular component is in the fixed position with the tubular guide member and the cutting element nested together.

FIG. 2D is a perspective image of the embodiment of FIG. 2A showing the modular component in the cutting position with the tubular guide member abutting the undersurface of the cutting element causing the tip of the cutting element to deploy through the slit of the cannula/needle.

FIG. 2E is a schematic showing the resulting fin that protrudes from the slot of the cannula/needle upon action of the modular components with each other.

FIG. 3A is a schematic of another embodiment of the present invention showing an instrument having a cannula/needle with at least one slit at its distal end and a stylet with a bullet-nose that extends beyond the distal tip of the cannula/needle. The handle of the cannula/needle and handle of the stylet mate to form one flattened surface that can be easily grasped by the user and impacted, if necessary.

FIG. 3B is a schematic of the distal end of the embodiment of FIG. 3A.

FIG. 3C is a schematic of an alternate embodiment of the embodiment of FIG. 3A showing an instrument having a cannula/needle and a stylet with a bullet-nose that extends beyond the distal tip of the cannula/needle. The instrument also includes an intermediary sheath that sits between the cannula/needle and the stylet, the sheath having at least one slit at its distal end.

FIG. 3D is a schematic of the distal end of the embodiment of FIG. 3C.

FIG. 3E is a cross-sectional image of the embodiment of FIG. 3C.

FIG. 3F is a schematic showing that the retraction of the bullet-nose tip of the stylet causes the sheath of FIG. 3C to flare outward at the location of the slits at the distal end of the sheath.

FIG. 4A is a schematic of another embodiment of the present invention showing a cannula/needle with at least one slot at its distal end that cooperates with a modular component within the hollow lumen of the cannula/needle of FIG. 2A. The modular component is in the fixed position with the tubular guide member and the stylet element nested together.

FIG. 4B is a schematic of the embodiment of FIG. 4A with the stylet in the extended position for creating a cavity of void in bone.

FIG. 5A is a schematic of another embodiment of the present invention showing a flexible stylet used in conjunction with a cannula/needle to create a void in bone.

FIG. 5B is a schematic of the embodiment of FIG. 5A with the flexible stylet inserted into the hollow lumen of the cannula/needle.

FIG. 5C is a schematic of the embodiment of FIG. 5A with the flexible stylet in the extended position. The tip of the stylet protrudes through the slot in the side wall of the cannula/needle.

FIG. 6A is a schematic of another embodiment of the present invention in which the stylet extends from the end opening of the cannula/needle.

FIG. 6B shows an alternate embodiment of the stylet of FIG. 6A in which the stylet has a blunt tip.

FIG. 6C shows the proximal handles of the embodiment of FIG. 6A/6B.

### DETAILED DESCRIPTION OF ILLUSTRATIVE EMBODIMENTS

The present invention is directed to instruments for use in bone; and in particular, instruments for creating voids, cavities and channels in bone. The present invention also relates to the use of instruments for bone biopsy and instruments for obtaining samples of bone tissue.

The instrument of the present invention may be used in a variety of medical procedures and in any type of bone including healthy cancellous and cortical bone, osteoporotic bone, metastatic/cancerous bone, bone subject to avascular necrosis, trauma and/or fracture. In a preferred embodiment, the instrument of the present invention includes a tool that creates access (e.g., a pathway) to bone and/or the interior of bone or is utilized in conjunction with such a tool. The instrument is capable of reducing a fracture in bone by compacting the bone. The compaction of bone, particularly cancellous bone, reduces its porosity and moves the particulate of bone from the center of the interior of the bone to the internal perimeter of bone (near the interior cortical layer) thereby creating a barrier which aids in the prevention of leaks upon the injection of material.

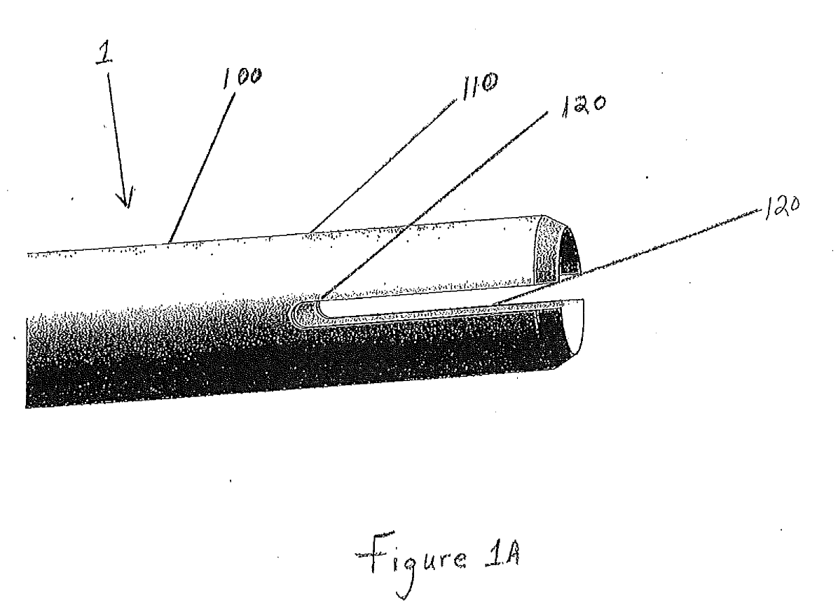

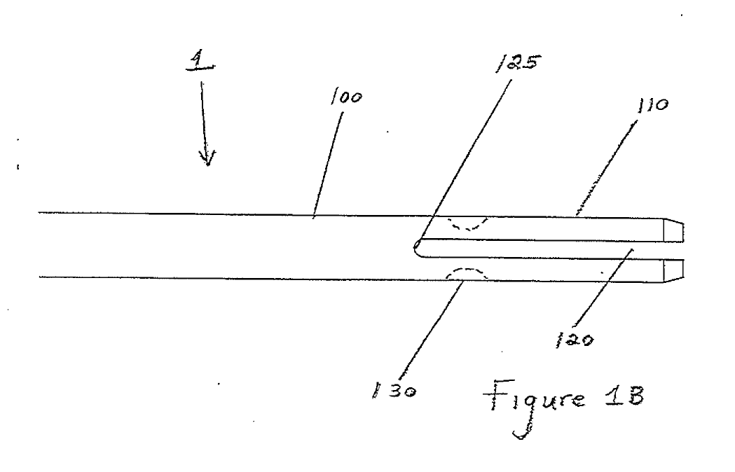

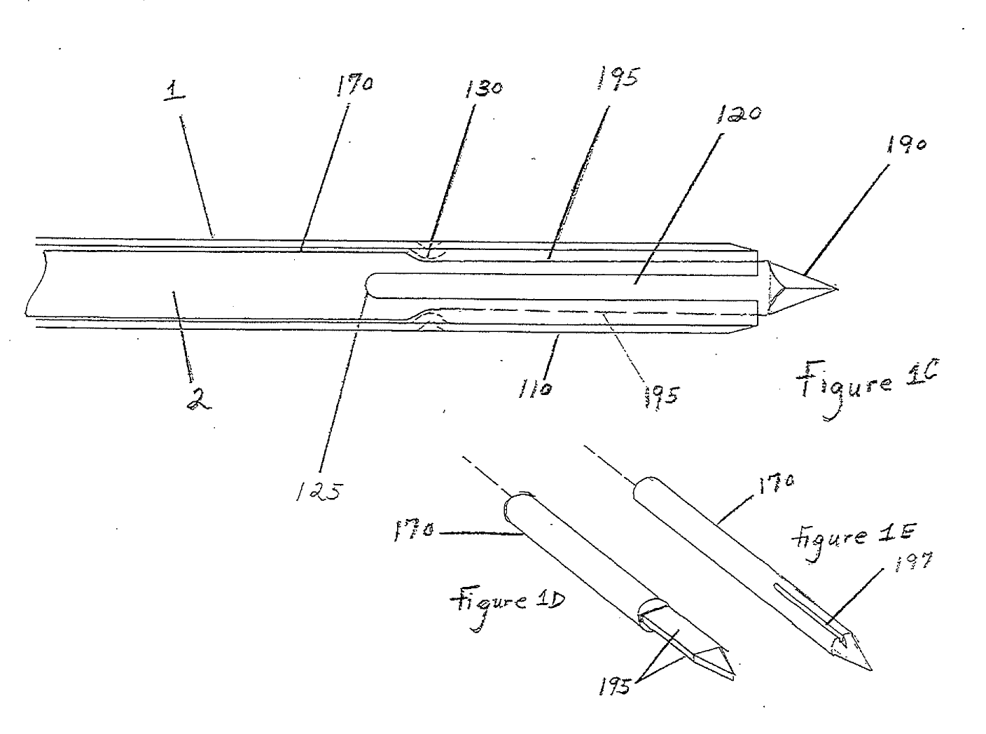

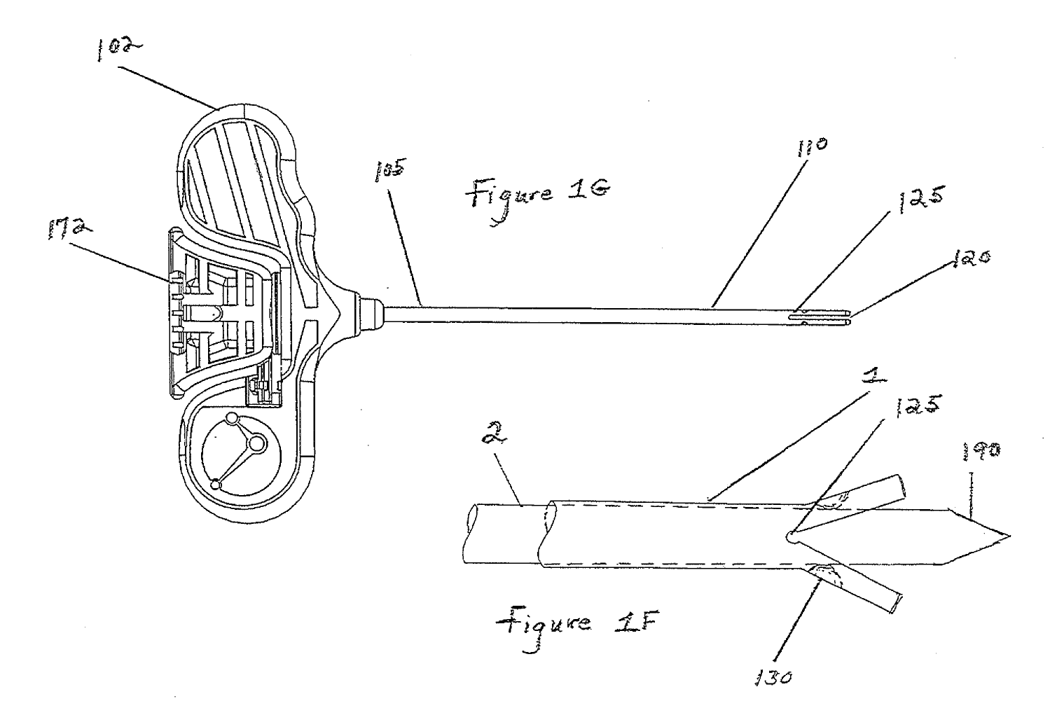

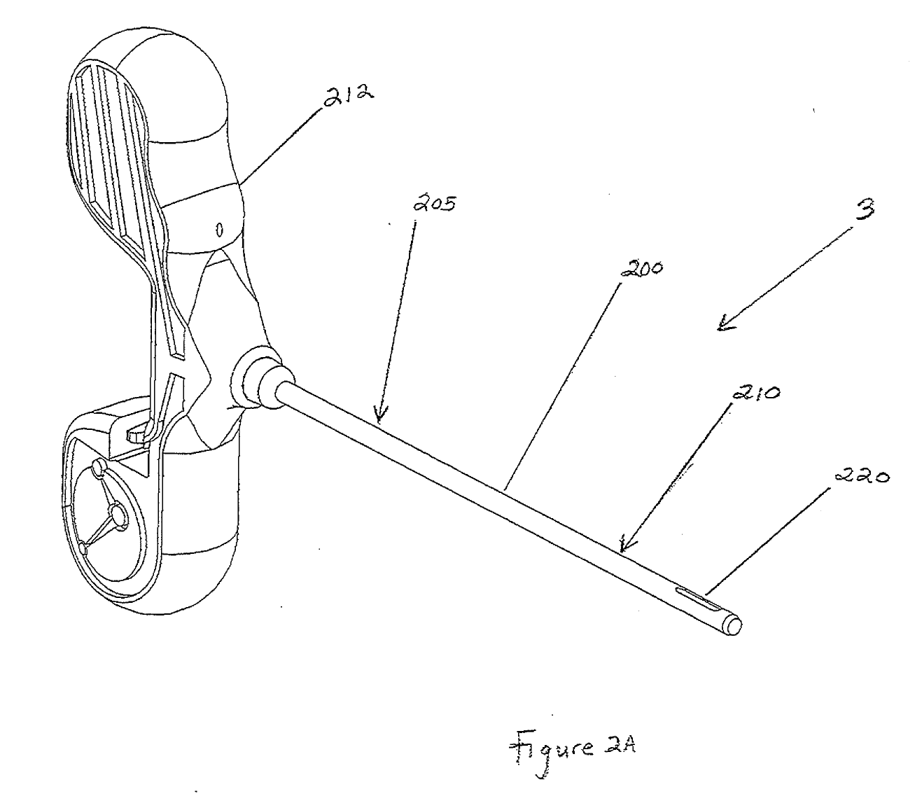

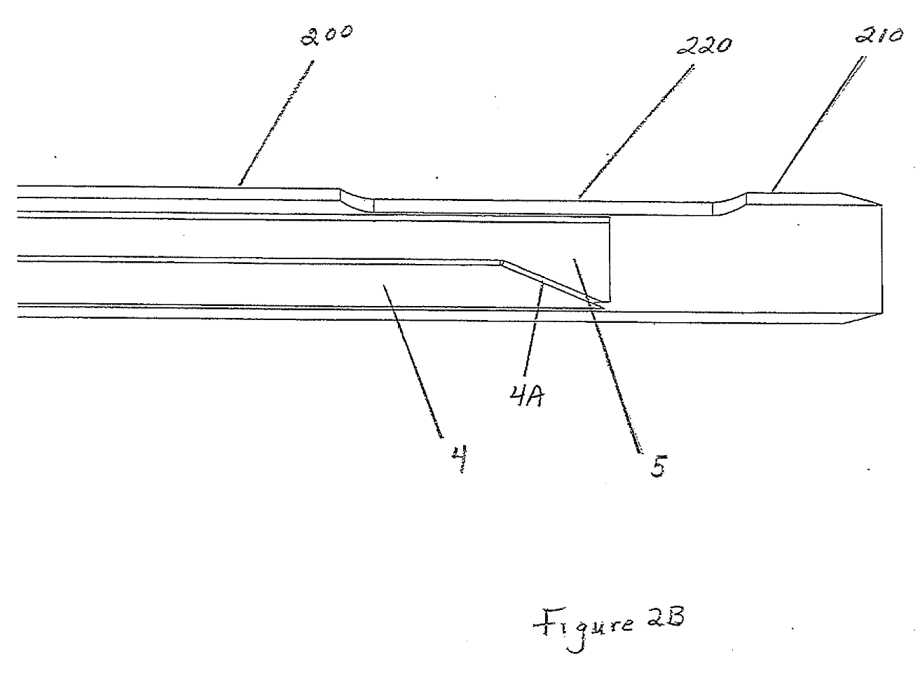

Flared Access Cannula with Slits: In one embodiment, the present invention provides for an instrument that includes an access cannula/needle 1 and a stylet 2. Upon engaging the components, the main access cannula/needle flares outward to create a void in the bone. As shown in FIGS. 1A-1C, the present invention includes a cannula/needle 1 and a stylet 2 which slidingly cooperates with the cannula/needle. The cannula/needle 1 is provided with a shaft 100 having proximal 105 and distal 110 ends and a longitudinally extending hollow lumen with an internal diameter. The diameter of the lumen of the cannula/needle is sized to receive the stylet. At least one slit 120 that extends through the wall thickness of the cannula/needle shaft is located at the distal end 110 of the cannula/needle. In a preferred embodiment, there are two or more opposing slits located at the distal end 110 of the cannula/needle that extend through the wall thickness of the cannula/needle shaft. The cannula/needle also comprises bumps 130 in the cannula/needle wall that protrude into the hollow lumen (FIG. 1B).

The stylet 2 also includes a shaft 170 having proximal 175 and distal 180 ends. The shaft of the stylet may be longer than the shaft of the cannula/needle. The distal tip 190 of the stylet may be beveled or diamond in shape for creating space in front of the cannula/needle. In one embodiment, the distal end of the shaft of the stylet also has a tapered region with flattened parallel faces 195 (FIG. 1D). In another embodiment, the distal end of the shaft of the stylet has opposing female grooves 197 or channels (instead of flattened parallel faces) (FIG. 1E).

In use, the stylet is inserted and advanced along the length of the cannula/needle. To create a cavity, the stylet is rotated from a first position to a second position. In the second position, the largest diametric region of the stylet 2 engages the bumps 130 on the inside of the hollow lumen of the cannula/needle which causes the distal end of the access cannula/needle to flare at the location of the distal slits (FIG. 1F). It should be noted that in the first position, the flattened parallel faces, or grooves/channels mate with the bumps on the inside of the hollow lumen of the cannula/needle (e.g., act similar to female counterparts for the male bumps so that the distal end of the cannula/needle is not caused to flare). To un-flare the cannula/needle, the stylet is rotated back to the first position.

The length of the slits in the cannula/needle in proportion to the total length of the shaft may vary. In certain embodiments, the ratio of the length of the slits to the length of the shaft of the cannula/needle is from about 1:15 to about 1:8. In a preferred embodiment, the ratio of the length of the slits to the length of the shaft of the cannula/needle is about 1:10. In addition to this ratio, the location of the bumps within the hollow lumen and size of the bumps can vary to achieve the desired angle of flare. In preferred embodiments, the bumps should be located distal to the vertex 125 of the slits. In embodiments where the desired flare angle is large, the bumps 130 may be located distal to and close to the vertex 125; while, in embodiments, in which a small angle of flare is desired, the bumps 130 may be located more distally away from the vertex 125. It should be understood various aspects of the instruments design will affect the angle of flare, including the length of the slits, the size of the bumps and the respective materials/material properties of the cannula/needle and stylet, however, it is preferred that various of these combinations will be employed to achieve an angle of flare of from about 5 degrees to about 80 degrees; more preferably from about 10 degrees to about 40 degrees.

In some embodiments, the stylet 2 is solid. Fixed to the proximal end (105, 175) of each of the cannula/needle and stylet is a handle (handle of cannula/needle 102, handle of stylet 172). In some embodiments, the handles of each nest together and are provided with a substantially lateral surface (flat, solid knob or pedestal) responsive to impact blows. In preferred embodiments, the handles are provided with markings, mechanical stops, or protrusions/indents to aid in the orientation of the stylet within the cannula/needle. Alternatively, adjustable stops may be provided on the stylet in order to control the axial travel and rotation of the stylet. The use of stops in this manner helps the clinician to predetermine the distance of travel of the stylet beyond the distal end opening of the cannula/needle, the angle of flare, and or the size of the cavity to be created. In addition, the adjustable stops may also allow the clinician to gradually create the cavity within controlled parameters.

In certain embodiments, the cannula/needle and stylet include gradations or marking along the length of each shaft to aid the clinician in determining the depth at which the components reach into the bone.

Various materials may be employed in the manufacture of the cannula/needle and stylet components. Generally, the cannula/needle and stylet are comprised of metals, such as stainless steel, titanium or metal alloy, which are rigid and readily visible by x-ray; but may also be comprised of plastic or polyimide, latex, silicone, vinyl or polymers other than those listed herein; or of ceramic. In some embodiments of the present invention, the components are comprised of nitinol, or any other “shape memory” alloy. In certain embodiments, the stylet may be constructed using standard flexible, medical grade plastic materials, like vinyl, nylon, polyethylenes, ionomers, polyurethane, and polyethylene teraphthalate.

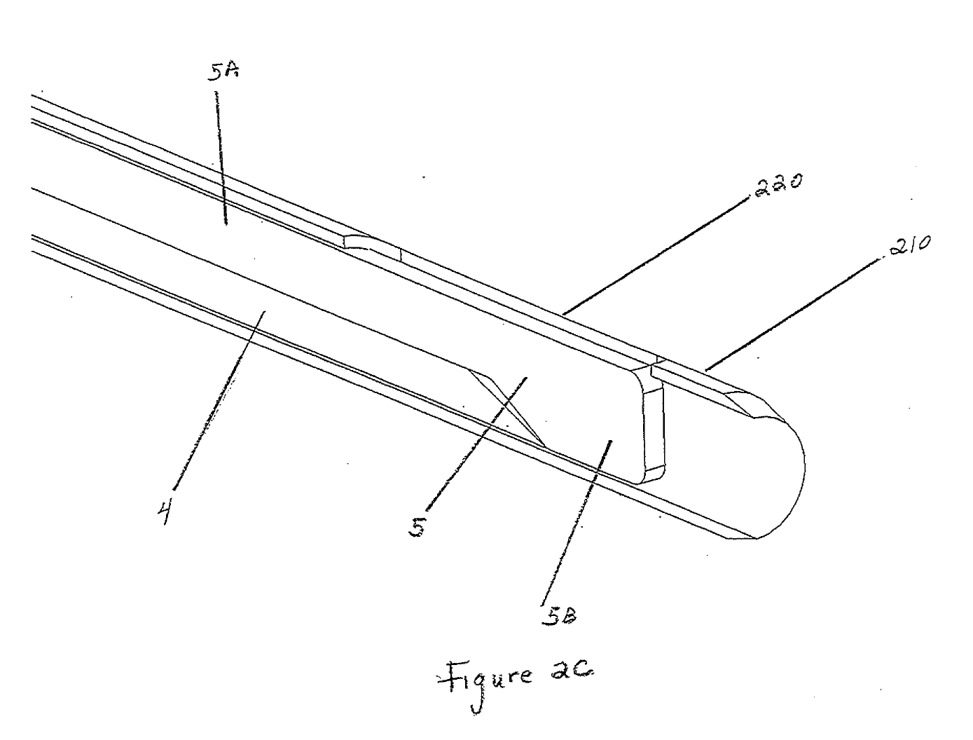

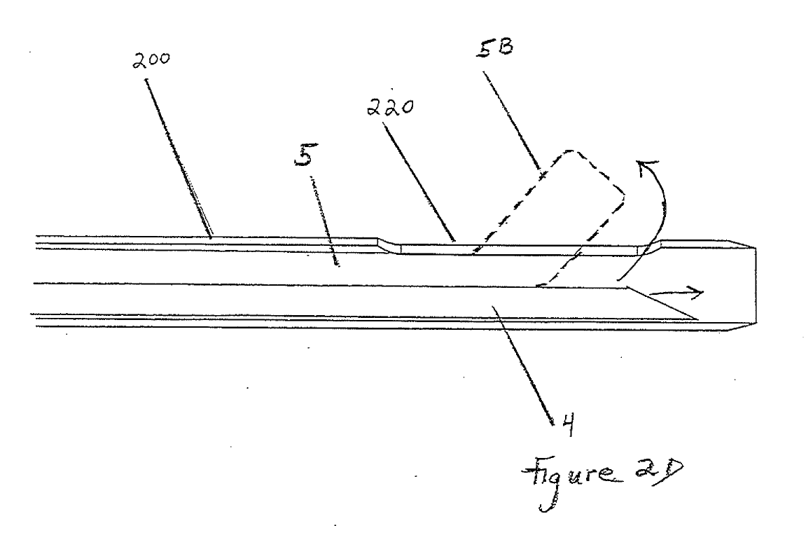

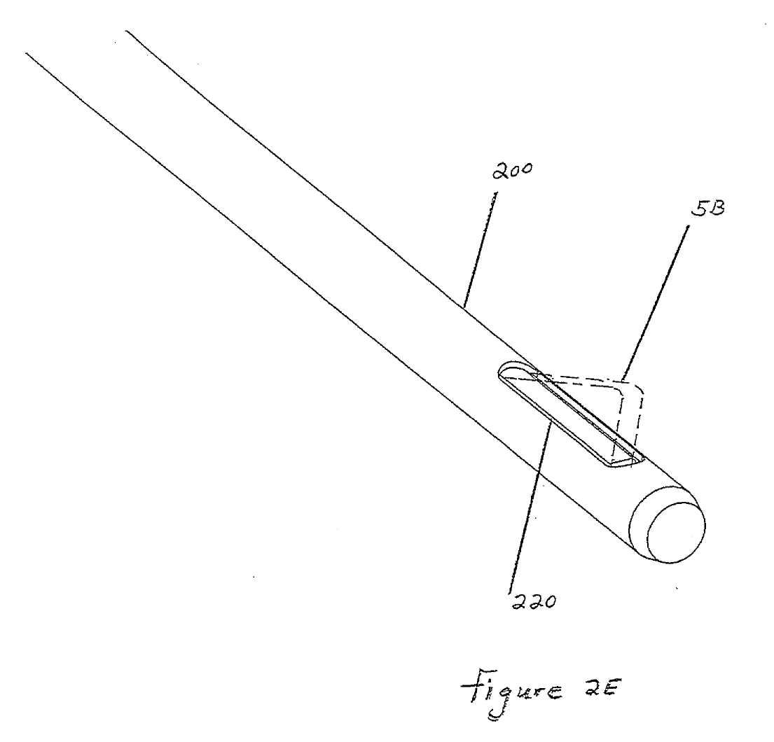

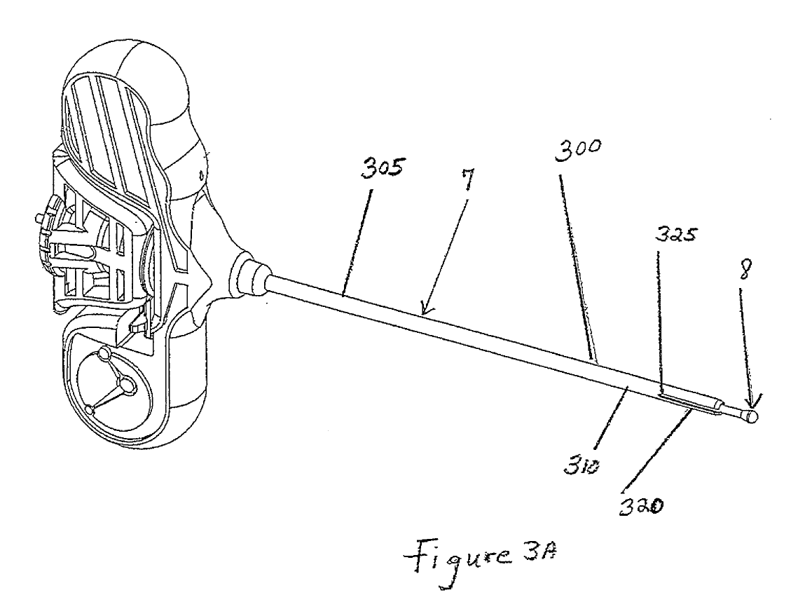

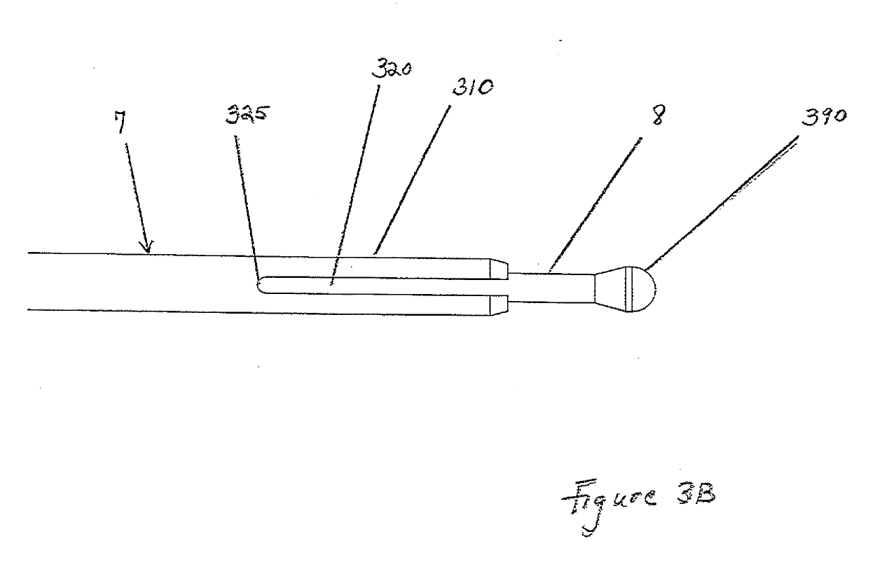

Access Cannula with Slot for Fin Protrusion: In another embodiment, the present invention provides for an instrument in which the access cannula/needle is provided with at least one slot at its distal end through which a modular component protrudes. As shown in FIGS. 2A-2E, the cannula/needle 3 is provided with a shaft 200 having proximal 205 and distal 210 ends and a longitudinally extending hollow lumen having an internal diameter. At least one slot 220 that extends through the wall thickness of the cannula/needle shaft is located at the distal end 210 of the cannula/needle 3.

A modular component cooperates with the cannula/needle 3 to create a void in the bone. The modular component includes a generally tubular guide member 4 and a cutting element 5 (FIGS. 2B, 2C). The tubular guide member is a generally solid member with a groove for mating with the cutting element. The cutting element 5 sits within the longitudinally extending groove of the tubular guide member 4. The cutting element is generally L-shaped with a long rectangular shaft 5A and fin tip 5B that angles outwardly away from the shaft 5A in a direction normal to the longitudinal axis of the shaft. The modular component includes two positions within the lumen of the cannula/needle—a first fixed position to enable the modular component to be slidingly advanced within and along the length of the hollow lumen (FIGS. 2B, 2C); and a second cutting position (FIG. 2D). In the fixed position, the cutting element sits within the groove of the generally tubular guide member 4 in such a manner that the tip of the cutting element overhangs the distal beveled end 4A of the tubular guide member 4. In the cutting position, the tubular guide member is advanced from a proximal to a distal direction along the undersurface of the tip 5B of the cutting element (FIG. 2D). When the components are in the cutting position, contact between the tubular guide member 4 and the angled fin tip 5B of the cutting element 5 deploys the fin tip 5B upward and through the slot 220 of the cannula/needle 3. Creation of a void in the bone may be effected in several ways when the cutting tip is in the cutting position. In a first manner, the cannula/needle and modular component are rotated as a unit to effect cutting; in a second manner, the tubular component is caused to translate back and forth (proximal to distal and then distal to proximal) to protrude and then extract the tip 5B of the cutting element 5 into and out of the bone. In a third manner, both rotation and translation as described above occur simultaneously to create a void in the bone. Alternatively, the cannula/needle 3 itself can be repositioned (e.g., moved into and out of the bone) with the fin tip 5B in the cutting position to compact bone and create a cavity; or the cannula/needle can be repositioned repeatedly with the fin tip down in the fixed position and then once repositioned, the fin tip is deployed in the cutting position.

The edge of the tip of the cutting element may be beveled, rounded, blunt, sharpened or have a roughened or textured surface or surface with protrusions to affect the desired cutting.

The diameter of the lumen of the cannula/needle is generally large enough to accommodate insertion of the modular component down the length of the cannula/needles. In preferred embodiments, the cannula/needle diameter ranges from about an 11 gauge to about an 8 gauge diameter; and the shaft length of the cannula/needle varies from about 4 inches to about 8 inches, and more preferably from about 4 inches to about 6 inches.

The proximal ends of each of the cannula/needle and stylet are provided with means for aiding in the alignment of the two components to insure that the tip of the cutting element protrudes from the slot of the cannula/needle in use. The means may include markings, cooperating protrusions/indents, mechanical stops or the like, as described above.

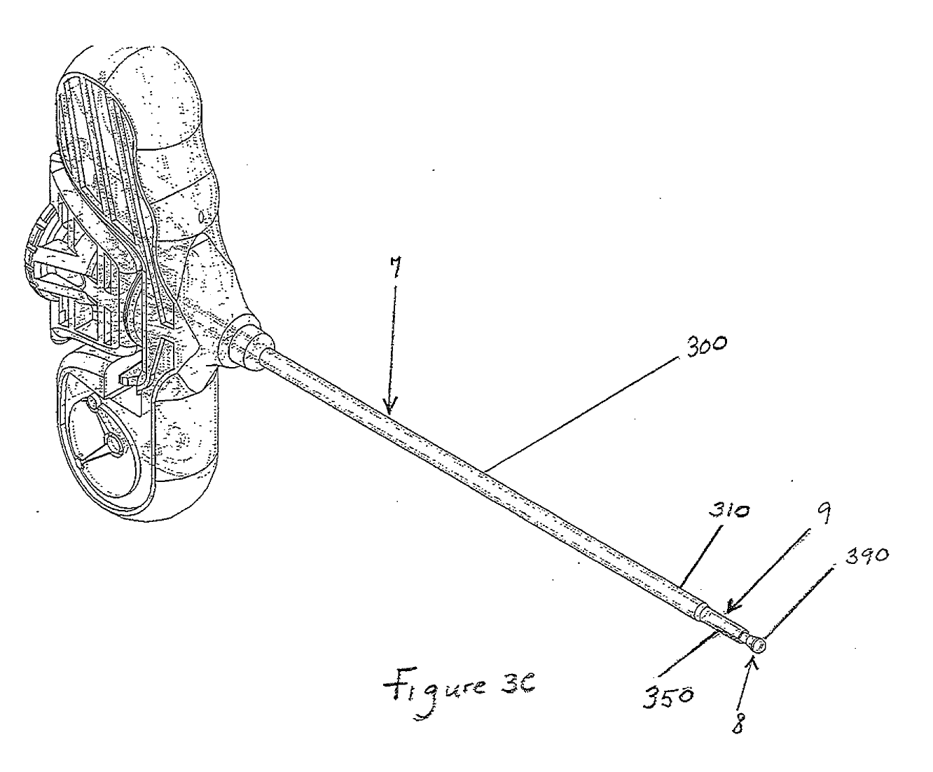

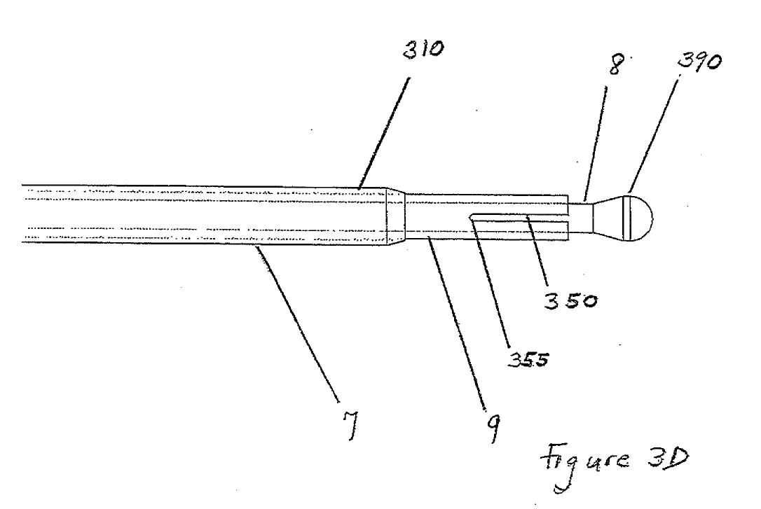

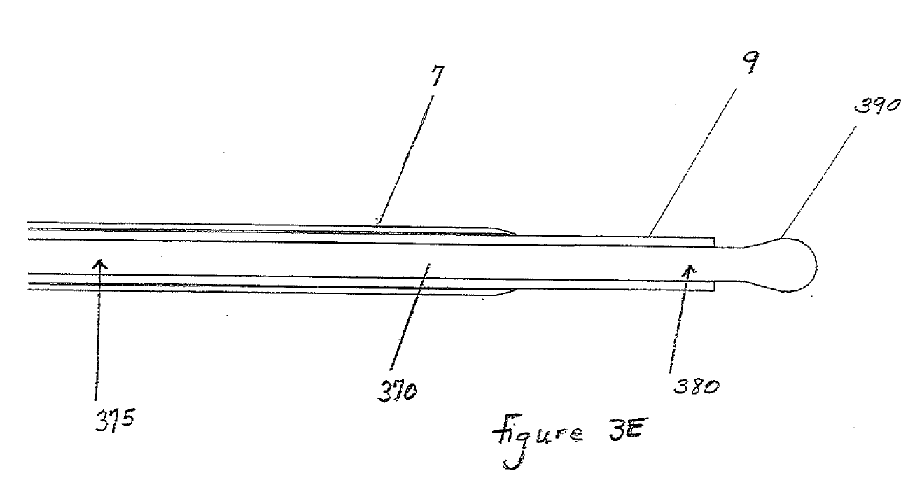

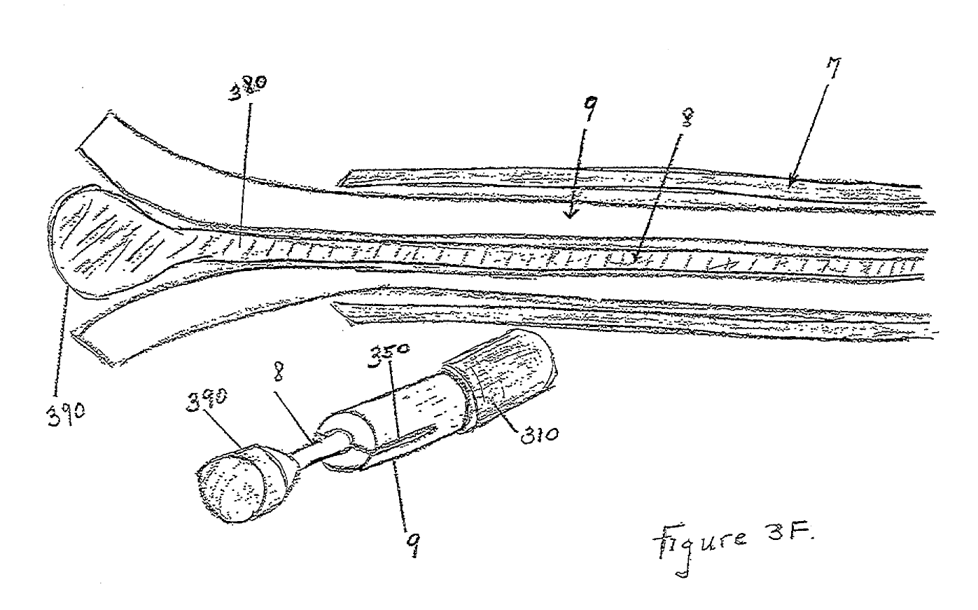

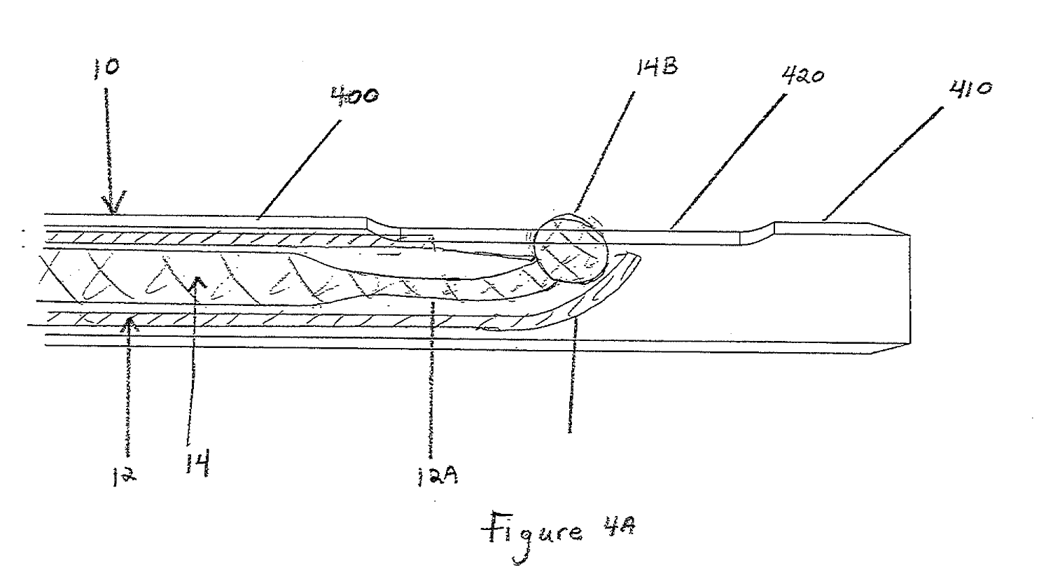

Flared Sheath/Access Cannula with Slits and Bullet-Nose Stylet: In another embodiment, the present invention provides for an instrument that includes an access cannula/needle 7 and a bullet-nose stylet 8. As shown in FIGS. 3A-3F, the cannula/needle 7 is provided with a shaft 300 having proximal 305 and distal 310 ends and a longitudinally extending hollow lumen with an internal diameter. At least one slit 320 (with vertex 325) that extends through the wall thickness of the cannula/needle shaft 300 is located at the distal end 310 of the cannula/needle. In a preferred embodiment, there are two opposing slits located at the distal end of the cannula/needle that extend through the wall thickness of the cannula/needle shaft.

The stylet 8 also includes a shaft 370 having proximal 375 and distal 380 ends. The distal tip of the stylet 8 has a bullet-nose 390. The bullet-nose tip 390 of the stylet extends past the end of the cannula/needle. The retraction of the stylet 8 back from a distal to a proximal direction, engages the opening of the cannula/needle which causes the split distal end 310 of the access cannula/needle 7 to flare. In certain embodiments (FIGS. 3C-3E), instead of the access cannula/needle 7 having slits, a sheath 9 that serves as an intermediary between the access cannula/needle 7 and stylet 8 (and extends out and beyond the end of the cannula) is provided with slits 350 (with vertex 355) at its distal end. In this manner, retraction of the stylet 8 from a distal position to a proximal position causes the sheath 9 to flare upon entry of the stylet tip 390 into the sheath distal opening. In alternate embodiments of the present invention, the end (tip) of the stylet is not bullet-nose but rather of any varying shape as long as the overall size of the shape is larger than the internal diameter of the sheath or cannula/needle.

It should be understood, as with the embodiment of FIGS. 1A-1G, the length of the slits in the cannula/needle or sheath in proportion to the total length of the shaft may vary. In certain embodiments, the ratio of the length of the slits to the length of the shaft of the respective component is from about 1:15 to about 1:8. In a preferred embodiment, the ratio of the length of the slits to the length of the shaft of the respective component is about 1:10. In addition to this ratio, the size of the bullet-nose tip (e.g., the diameter of the bullet-nose or size of the tip in a different shape/form), as well as the respective materials/material properties of the cannula/needle, sheath and stylet will affect the angle of flare, however, it is preferred that various of these combinations will be employed to achieve an angle of flare of from about 5 degrees to about 80 degrees; more preferably from about 10 degrees to about 40 degrees.

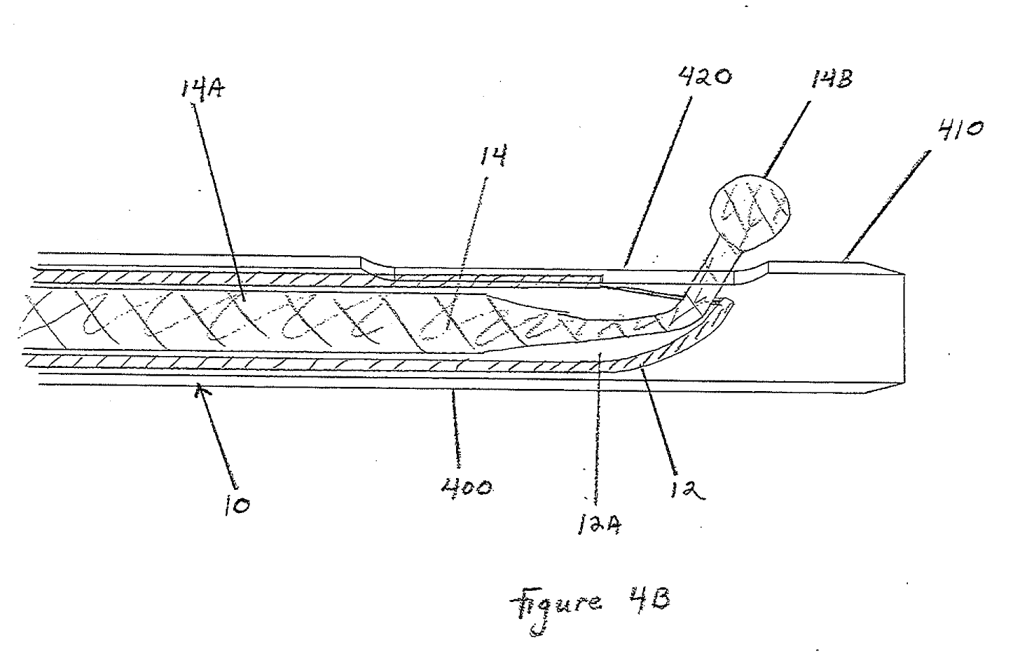

Bendable Stylet: In yet another embodiment, the present invention provides for an instrument in which the access cannula/needle is provided with at least one slot at its distal end through which a modular component protrudes. As shown in FIGS. 4A-4B, the cannula/needle 10 is provided with a shaft 400 having proximal 405 and distal 410 ends and a longitudinally extending hollow lumen having an internal diameter. At least one slot 420 that extends through the wall thickness of the cannula/needle shaft is located at the distal end 410 of the cannula/needle.

The modular component cooperates with the cannula/needle 10 to compact and create a void in the bone. The modular component includes a tubular guide member 12 and a stylet 14. The tubular guide member 12 is a solid member with a channeled recessed groove 12A (along its length) for mating with the stylet 14. The stylet 14 sits within the recessed groove 12A of the tubular guide member 12. The stylet 14 includes a long flexible shaft 14A and tip 14B that angles outwardly away from the longitudinal axis of the shaft. The modular component includes two positions within the lumen of the cannula/needle 10—a first fixed position which enables the modular component to be slidingly advanced within and along the length of the hollow lumen (FIG. 4A); and a second extended position (FIG. 4B). In the fixed position, the stylet sits within the recessed groove 12A of the generally tubular guide member 12. In the extended position, the tubular guide member 12 is advanced from a proximal position to a distal position so that the tip 14B of the stylet 14 protrudes through the slot 420 of the cannula/needle 10. Creation of a void in the bone may be effected in several ways when the tip 14B is in the extended position. In a first manner, the cannula/needle 10 and modular component are rotated as a unit to effect cutting; in a second manner, the tubular component 12 is caused to translate back and forth (proximal to distal and then distal to proximal) to protrude and then extract the tip 14B of the stylet 14 into and out of the bone. In a third manner, both rotation and translation as described above occur simultaneously to create a void in the bone. Alternatively, the cannula/needle 10 itself can be repositioned (e.g., moved into and out of the bone) with the tip 14B in the extended position to compact bone and create a cavity; or the cannula/needle can be repositioned repeatedly with the tip 14B down in the fixed position and then once repositioned, the tip is deployed in the extended position.

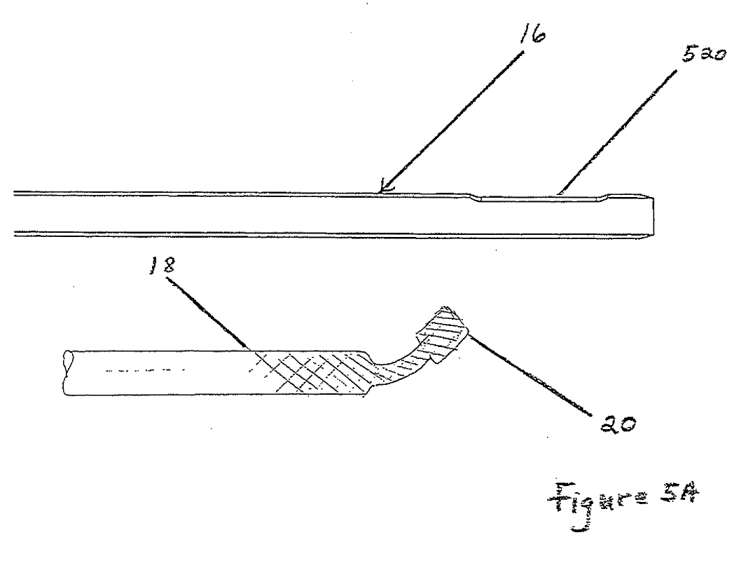

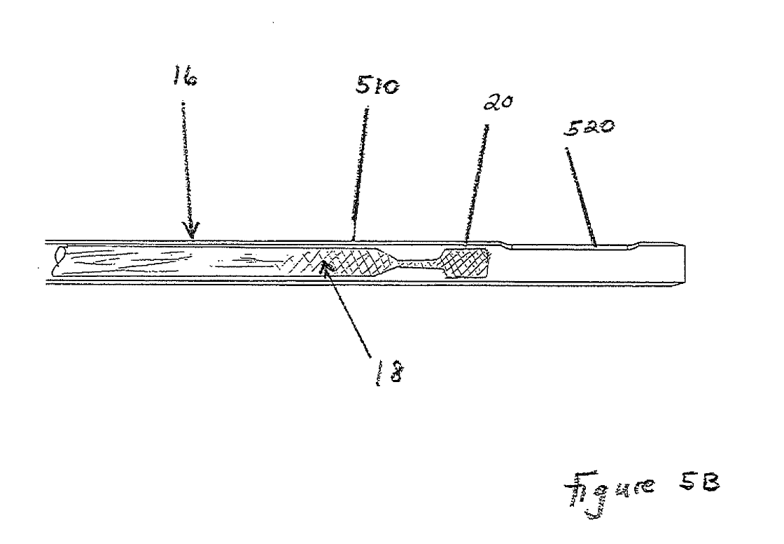

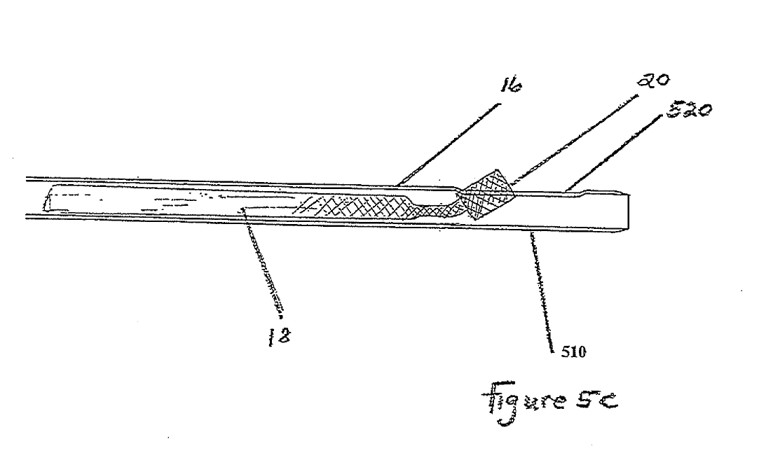

Similar to the embodiment shown in FIGS. 4A-4B, FIGS. 5A-5C show an embodiment of an instrument in which the access cannula/needle is provided with at least one slot at its distal end through which a stylet component protrudes. Unlike the embodiment of FIGS. 4A-4b, however, the embodiment shown in FIGS. 5A-5C does not utilize a tubular guide sheath. In this embodiment, a stylet 18 with a flexible tip 20 is actuated through a slot 520 in the distal end 510 of the access cannula/needle 16. Void creation in the bone may be affected in the same ways as described above with respect to FIGS. 4A-4B.

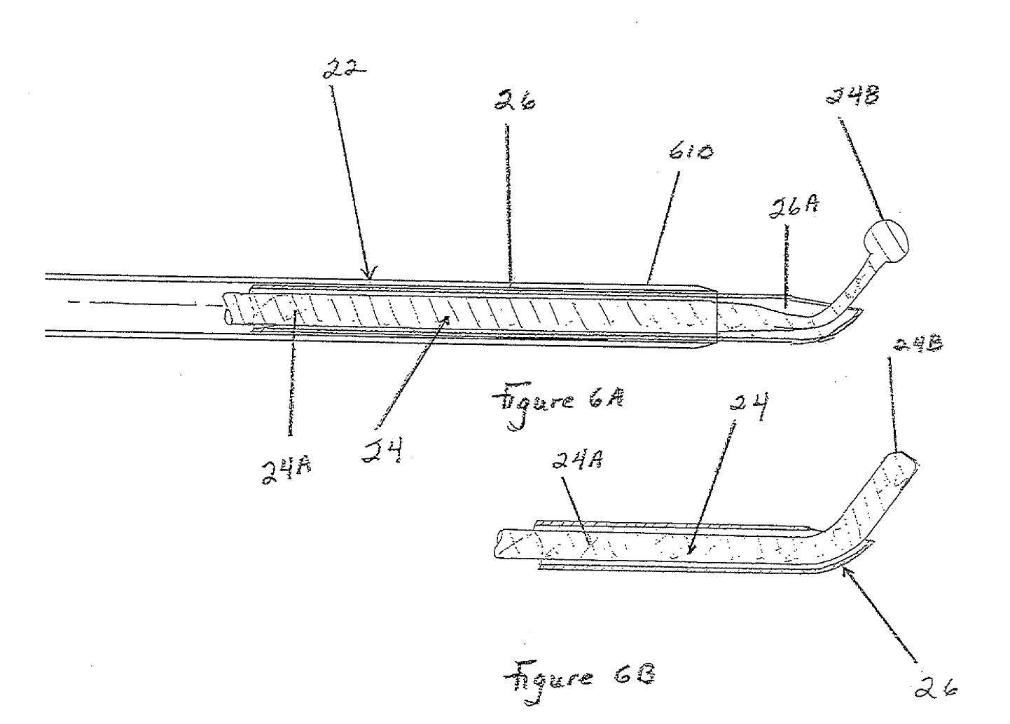

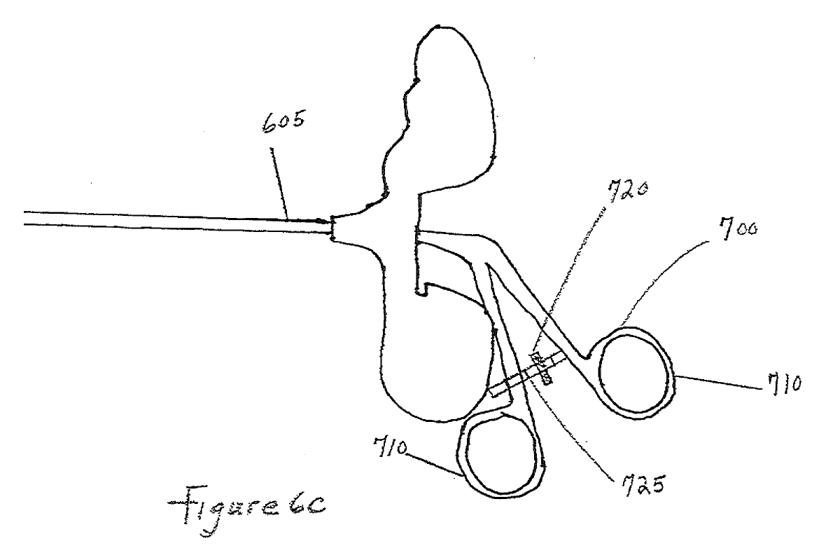

Similar to the embodiment shown in FIGS. 4A-4B, FIGS. 6A-6C show an embodiment of an instrument in which the access cannula/needle cooperates with a modular component to affect void creation. As shown in FIGS. 6A-6C, the cannula/needle 22 is provided with a shaft 600 having proximal 605 and distal 610 ends and a longitudinally extending hollow lumen having an internal diameter. The modular component cooperates with the cannula/needle 22 to compact and create multiple channels (in varying directions) in the bone. This directional channel creation aids in the dispersion, interdigitation and flow of material into the bone. The modular component includes a tubular guide member 26 and a stylet 24. In preferred embodiments, the tubular guide member 26 extends past the distal opening of the cannula/needle. The tubular guide member 26 is a solid member with a channeled recessed groove 26A (along its length) for mating with the stylet 24. The stylet 24 sits within the recessed groove 26A of the tubular guide member 26. The stylet 24 includes a long flexible shaft 24A and tip 24B that angles outwardly away from the longitudinal axis of the shaft. The modular component includes two positions within the lumen of the cannula/needle 22—a first fixed position which enables the stylet to be slidingly advanced within and along the length of the hollow lumen (not shown but similar to the position of the stylet of FIG. 4A); and a second extended position (FIGS. 6A and 6B). In the fixed position, the stylet 24 sits within the recessed groove 26A of the tubular guide member 26. In the extended position, the handle 700 of the modular component is actuated (FIG. 6C) so that the stylet 24 protrudes tangentially above and beyond the opening of the tubular guide member 26 through the end of the cannula/needle 22 (FIGS. 6A and 6B). Channels in the bone may be affected in several ways when the stylet tip 24B is in the extended position. In one preferred manner, the modular component is cycled from the fixed position to the extended position. In another manner, the modular component is cycled from the fixed position to the extended position and rotated in a controlled manner. In a third manner, both rotation and extension as described above occur simultaneously to create the channels in the bone. Alternatively, the cannula/needle 22 itself can be repositioned (e.g., moved into and out of the bone) with the tip 24B in the extended position to compact bone and create a cavity; or the cannula/needle can be repositioned repeatedly with the tip 24B down in the fixed position and then once repositioned, the tip is placed in the extended position. The tip of the present invention may be rounded or blunt or of any desired shape that will produce the resultant channel shape.

Although there are many handle arrangements that could be employed with the present invention, one type of arrangement preferred for deploying the stylets of FIGS. 6A and 6B is shown in FIG. 6C. In this embodiment, the handle arrangement 700 for the modular component includes means for actuating the stylet into the extended position by squeezing finger tabs 710. An adjustable stop 720 is provided on the handle in order to control the tangential travel of the stylet. The stop 720 may be marked in increments 725 which correspond to distance of travel of the stylet. The use of stops in this manner helps the clinician to predetermine the distance of travel of the stylet beyond the distal end opening of the tubular guide member and/or the angle of flare and/or the size of the channels to be created. In preferred embodiments, the end of the tubular guide member extends from about 1 cm to about 3 cm beyond the end of the cannula/needle and the stylet can be extended from about 0.5 cm to about 2 cm beyond the end of the tubular guide member.

Various materials may be employed in the manufacture of the cannula/needle, stylet and tubular components described herein. In certain embodiments, the components are comprised of metals, such as stainless steel, titanium or metal alloy; but may also be comprised of plastic or polyimide, latex, silicone, vinyl or polymers other than those listed herein; or of ceramic. In some embodiments of the present invention, the components are comprised of nitinol, or any other “shape memory” alloy. In certain embodiments, the stylet and/or tubular guide member may be constructed using standard flexible, medical grade plastic materials, like vinyl, nylon, polyethylenes, ionomers, polyurethane, and polyethylene teraphthalate.

The present invention also provides methods for creating cavities, voids and channels in bone and methods for reducing fractures in bone. While the present invention envisions many methods of creating cavities, voids and channels in bone using the instruments of the present invention one method provides for void creation comprising the steps of: establishing a percutaneous access path in bone using a needle, wherein the needle comprises a hollow lumen and at least one slit located at the distal end of the needle through a wall thickness of the needle and further comprises at least one protrusion extending into the hollow lumen of the needle from an internal wall surface of the needle, sliding a stylet down the hollow lumen of the needle; and rotating the stylet so that when an outer wall portion of the stylet engages the at least one protrusion, the distal end of the needle flares with a flare angle to create a cavity in bone. In one embodiment, for void or cavity creation, the angle of flare is from about 5 to about 40 degrees. In other embodiments in which fracture reduction is desired, the flare angle may be from about 10 to about 80 degrees.

In certain embodiments, the instruments described herein may be employed in conjunction with other instruments. In certain embodiments, the instruments of the present invention cooperate with a micro-reamer, such as that disclosed in U.S. Pat. No. 7,544,196 assigned to the assignee of the present invention and hereby incorporated by reference in its entirety. For instance, the instruments of the present invention may be used to first compact and create a channel in the bone and then the micro-reamer could be used to further enlarge the cavity and/or remove bone.

This present invention also relates to device combinations and packaged kits. These comprise one or more cannulae/needles adapted for accessing said intraosseous space as described herein; one or more stylets insertable into the hollow cavity of the cannula and being movable therein to advance the cannula into position, to compact the bone and/or to create a cavity in the bone. The kit may also include stylets capable of removing bone tissue. The kits and systems preferably also have one or more catheters that are insertable into the cannulae, and a system for delivery of aliquots of said restorative or injectable composition into the intraosseous space via the catheters.

The disclosures of each and every patent, patent application, and publication cited herein are hereby incorporated herein by reference in their entirety. Although the present invention has been described with reference to instruments for use in bone, it should be understood that aspects of the present invention, such as the methods of making the components of the instruments, and their methods of use with a restorative bone composition, are not limited to the particular embodiments disclosed. While the present invention has been particularly shown and described with reference to the presently preferred embodiments thereof, it is understood that the invention is not limited to the embodiments specifically disclosed herein. Numerous changes and modifications may be made to the preferred embodiment of the invention, and such changes and modifications may be made without departing from the spirit of the invention. It is therefore intended that the appended claims cover all such equivalent variations as they fall within the true spirit and scope of the invention.

What is claimed is:

1. An instrument for use in bone for creating a void in the bone comprising means for accessing an interior of the bone and actuating means for cooperating with the means for accessing the interior of bone to create a void in bone.

2. The instrument of claim 1, wherein the means for accessing the interior of bone is a cannula.

3. The instrument of claim 1, wherein the means for accessing the interior of bone is a needle.

4. The instrument of claim 1 wherein the means for accessing the interior of bone is a cannula with a hollow lumen and at least one slit located at the distal end of the cannula.

5. The instrument of claim 1 wherein the means for accessing the interior of bone is a needle with a hollow lumen and at least one slit located at the distal end of the needle.

6. The instrument of claim 5 wherein the needle further comprises protrusions extending into the hollow lumen of the needle from an internal wall surface of the needle and wherein the actuating means contacts the protrusions to flare the distal end of the needle to create a void in bone.

7. An instrument for use in bone for creating channels in the bone comprising means for accessing an interior of the bone and a modular component for cooperating with the means for accessing the interior of bone to create a void in bone.

8. The instrument of claim 7, wherein the means for accessing the interior of bone is a cannula.

9. The instrument of claim 7, wherein the means for accessing the interior of bone is a needle.

10. The instrument of claim 7 wherein the modular component includes a tubular guide and a stylet with a flexible tip, and wherein the stylet fits within a longitudinal groove of the tubular guide.

11. The instrument of claim 10 wherein the modular component fits through a hollow lumen of the means for accessing the interior of bone and has a length that is longer than the means for accessing the interior of bone.

12. A method for creating a cavity in bone comprising the steps of:
establishing a percutaneous access path in bone using a needle, wherein the needle comprises a hollow lumen and at least one slit located at the distal end of the needle through a wall thickness of the needle and further comprises at least one protrusion extending into the hollow lumen of the needle from an internal wall surface of the needle,
sliding a stylet down the hollow lumen of the needle; and
rotating the stylet so that when an outer wall portion of the stylet engages the at least one protrusion, the distal end of the needle flares with a flare angle to create a cavity in bone.

13. The method of claim 12 wherein the flare angle is between about 5 degrees and about 40 degrees.

14. The method of claim 12 wherein the flare angle is between about 15 and 25 degrees.

15. A method for reducing a fracture in bone comprising the steps of:
establishing a percutaneous access path in bone using a needle, wherein the needle comprises a hollow lumen and at least one slit located at the distal end of the needle through a wall thickness of the needle and further comprises at least one protrusion extending into the hollow lumen of the needle from an internal wall surface of the needle,
sliding a stylet down the hollow lumen of the needle; and
rotating the stylet so that when an outer wall portion of the stylet engages the at least one protrusion, the distal end of the needle flares with a flare angle to create a cavity in bone.

16. The method of claim 15 wherein the flare angle is between about 10 degrees and about 80 degrees.

17. The method of claim 15 wherein the flare angle is between about 15 and 55 degrees.
